(19) United States
(12) Patent Application Publication
(10) Pub. No.: US 2009/0204061 A1
(43) Pub. Date: Aug. 13, 2009

(54) METHOD AND APPARATUS FOR TREATING CELLULITE

(75) Inventors: James J. Pomposelli, Carlisle, MA (US); Susan A. Pasanen, North Andover, MA (US); Nancy C. Donahue, Lowell, MA (US); Heidi B. Kummer, Carlisle, MA (US)

Correspondence Address: LANDO & ANASTASI, LLP, ONE MAIN STREET, SUITE 1100, CAMBRIDGE, MA, 02142, US

(73) Assignee: BELLECORE, LLC, Lowell, MA (US)

(21) Appl. No.: 12/369,916

(22) Filed: Feb. 12, 2009

Related U.S. Application Data
(60) Provisional application No. 61/065,452, filed on Feb. 12, 2008.

(51) Int. Cl. A61H 7/00; A61M 35/00

(52) U.S. Cl. 604/22; 601/89

(57) ABSTRACT

A device and a method for treating cellulite is disclosed. An oscillation therapy device includes a body, a motor, a drive shaft powered by the motor, and a contact surface coupled to the drive shaft, which may be substantially enclosed in a covering adapted to frictionally engage human skin. The motor and drive shaft drive the contact surface to impart an oscillating motion to the contact surface and to skin in contact with the surface.

### RELATED APPLICATIONS

This application claims priority under 35 U.S.C. § 119(e) to U.S. Provisional Application Ser. No. 61/065,452, entitled “METHOD AND APPARATUS FOR TREATING CELLULITE,” filed on Feb. 12, 2008, which is herein incorporated by reference in its entirety.

### BACKGROUND OF INVENTION

1. Field of Invention

Embodiments of the present invention relate to methods and apparatus for providing oscillating mechanical energy to tissue and for treating tissue cellulite.

2. Discussion of Related Art

Gynoid lipodystrophy, or cellulite, affects 80-85% of women and results in the abnormal appearance of skin (commonly referred to as “Peau d'orange” or orange peel skin). The thighs, buttocks and lower back are the most commonly affected areas of the body. Although gynoid lipodystrophy is generally found only in women, under certain hormonal conditions such as deficiency in androgens or high levels of estrogens, men can also develop cellulite.

Human skin is made up of three distinct layers which include the epidermis, dermis, and hypodermis. The hypodermis contains the adipose tissue (fat) which is anchored by fibrous connective tissue arches to the dermis above and the anterior fascia of the muscle below. These arches compartmentalize the fat into three dimensional chambers measuring about 0.5-1.0 cm across. Since the anchor points and arches do not allow fat cells to expand beyond a certain volume, cellulite becomes apparent when the chamber volume exceeds its maximal capacity through either increased fat cell growth or edema (swelling). In addition, excessive fat deposits exert pressure on the small blood and lymphatic vessels, preventing them from cleansing the fat cells of toxins. Retained toxins may damage the cell walls, causing inflammation and thickening and hardening of the surrounding tissues (scar formation) which may also exacerbate cellulite appearance. Since cellulite is the result of mechanical forces in fat caused by hypertrophy and swelling, it is not considered a pathological condition. Weight loss alone does not predictably improve the appearance of cellulite, and in some individuals actually makes it look worse.

Historically, a common treatment of cellulite has involved the application of various cream products massaged onto the affected area. Despite the popularity of cellulite creams, the inventors are aware of no prospective randomized trials to date that have demonstrated efficacy of such creams. Some success in reducing the hardness and thickening of cellulite tissue has been reported with deep tissue massage therapy using macro-mechanical forces.

An accepted concept in treating human cellulite focuses on reducing tissue edema and increasing connective tissue elasticity. For this reason, massage therapy (the application of macro-mechanical forces), which softens the tissue and increases tissue perfusion is one therapeutic strategy. However, this technique is time consuming and has limited effectiveness since the application of forces is not standardized and is highly dependant on the quality of the massage therapist. Too forceful of an application of massage therapy can result in tissue injury, bruising, increased tissue edema, pain, and scarring of the traumatized tissue. The application of too little force may fail to achieve the desired effect. In addition, this modality is rather expensive and time consuming and requires multiple treatments to achieve and maintain the desired effect.

Another strategy in treating cellulite is to directly treat the affected tissues at the cellular level. Micro-mechanical forces such as high frequency ultrasound, radio frequency, or laser therapy may be applied in an attempt to increase tissue perfusion and reduce edema by subjecting the affected tissues to low amplitude repetitive mechanical forces. While these therapies also increase tissue perfusion and thereby reduce edema, they provide very little effect in terms of reducing tissue stiffness or increasing elasticity of connective tissue surrounding the fat cells. Regardless, these technologies are safe and somewhat effective, but are expensive and need to be applied professionally.

The inventors have discovered a method for the treatment of cellulite which combines the benefits of macro and micro-mechanical forces and that can be applied in a standardized fashion at home while being safe, effective, and affordable.

### SUMMARY OF INVENTION

A novel apparatus for and an approach to providing mechanical oscillation therapy is presented that may be used for treating human cellulite with a device that may be suitable for in-home use. This approach combines the benefits of macro and micro-mechanical forces to significantly reduce soft tissue edema, and increase fibrous connective tissue elasticity and blood flow. The net effect is an improvement in the feel and look of tissue characterized by cellulite.

Significant reduction of human cellulite can be achieved by treatment with a device that operates according to the embodiments disclosed herein. High frequency orbital oscillation combined with the massaging motion of some embodiments of the device disclosed significantly reduces tissue edema by increasing tissue perfusion and also increases connective tissue elasticity by mechanically stretching the fibrous arches in the hypodermis. The net result is a smoother, less dimpled, and softer skin. The palpable effect of smoother skin may be achieved after just a few applications while visual reduction of dimpled skin may take approximately 2-3 weeks of therapy.

Repeated application of random orbital oscillating energy on cellulite can result in fat cells being broken down while circulation of blood and lymph is greatly enhanced. The thickened and tight fibrous arches are stretched or even disrupted by the shear forces generated, preventing them from pulling the skin down to form dimples in skin. The net effect is a thinning of the fat in these areas providing a more durable and favorable cosmetic appearance.

The application of random orbital oscillating mechanical energy not only has the benefit of reducing the appearance of cellulite and adiposity but can also be used to increase tissue microcirculation (blood flow). The use of random orbital oscillating mechanical energy to increase microcirculation and tissue oxygenation may be performed as part of a pre-workout warm-up or a preparation for athletic events or exercise. Increased tissue perfusion can also help speed recovery after injury by enhancing lymphatic drainage and thus decreasing abnormal water retention (tissue swelling).

In an embodiment of the present invention, a mechanical oscillation treatment device comprises a body, a motor positioned within said body, and a drive shaft coupled to the motor. The motor is adapted to power the drive shaft. The drive shaft transmits energy to a contact surface coupled to the drive shaft. The contact surface is adapted to frictionally engage human skin. The motor and drive shaft are adapted to impart an oscillating motion to the contact surface. In some embodiments the motor and drive shaft are adapted to impart a random orbital oscillating motion to the contact surface. In some embodiments, the frequency of random orbital oscillating energy imparted to the contact surface is between about 2000 and about 3000 Hz, and in some embodiments, is about 2400 Hz. In some embodiments, the random orbital oscillating motion has an orbit diameter of from about 8 to about 12 mm, and in some embodiments, about 10.2 mm.

In accordance with some embodiments, the mechanical oscillation treatment device further comprises a pressure sensor coupled to the contact surface. The pressure sensor is in some embodiments coupled to a pressure indicator and/or a shutoff configured to be activated when the pressure sensor reads a pressure exceeding a pre-set pressure value. The pre-set pressure value may be in a range from about 10 to about 15 pounds.

In some embodiments, the contact surface of the mechanical oscillation treatment device comprises a multilayered rigid disk component. The multilayered rigid disk component includes materials such as polyurethane or cast aluminum followed by a layer of cellular or foam rubber, which may be about one inch thick, and finally by a surface cover comprising cotton, lambswool, or polyester. The multilayered rigid disk component will not bend on application of about 25 pounds of pressure to the device. In some embodiments, the surface cover may be disposable or washable. In some embodiments, the surface cover includes an elastic band to facilitate securing the cover to the device. In some embodiments, the covering comprises a polymeric material with massaging protrusions or bristles.

In some embodiments, the mechanical oscillation treatment device includes a heating means in thermal contact with the contact surface.

A method of applying oscillating mechanical energy to human tissue according to an embodiment of the present invention comprises contacting an area of tissue characterized by cellulite with the contact surface of a mechanical oscillation treatment device, and applying power to the motor of said device while said contact surface is in contact with an area of human tissue. In some embodiments of the method, orbital oscillating energy is applied to the tissue by oscillating the contact surface of a device in a direction approximately parallel to surface of the tissue in an orbital motion to induce shearing and stretching forces in the tissue. The oscillating energy applied to the tissue is, in some embodiments, random orbital oscillating energy.

In some embodiments of the method, the oscillating energy is applied to one or more areas of human tissue affected by cellulite for approximately 5-10 minutes, using continuous active movement of the device. In some embodiments, this application is performed daily, an average of twice per day.

In some embodiments, the oscillating energy is applied to an area of tissue to be treated with a contact pressure of from about 4 to about 12 pounds between the device and the area of affected tissue.

An embodiment of the method of applying oscillating energy to human tissue further comprises applying a cellulite treatment enhancement compound to the area of tissue after contacting the area of tissue with the contact surface of the device for treating cellulite. The cellulite treatment enhancement compound may comprise an analgesic, a warming solution, or a moisturizer.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is an exploded view of an embodiment of a mechanical oscillation therapy device in accordance with the present disclosure;

FIG. 2 is a side elevational view of an embodiment of the mechanical oscillation therapy device of FIG. 1 from the front side;

FIG. 3 is a top plan view of an embodiment of the oscillation therapy device;

FIG. 4 is a side elevational view of an embodiment of the oscillation therapy device of FIG. 1 from the right side;

FIG. 5 is a cross sectional view of the oscillation therapy device of FIG. 1 through line A-A of FIG. 4;

FIG. 6A is a side view of a motor included in an embodiment of the oscillation therapy device of FIG. 1;

FIG. 6B is a front view of a motor included in an embodiment of the oscillation therapy device of FIG. 1;

FIG. 6C is a rear view of a motor included in an embodiment of the oscillation therapy device of FIG. 1;

FIG. 7 is a flow chart of a method of treating cellulite according to one method disclosed herein;

FIG. 8 is a flow chart of another method of treating cellulite according to one method disclosed herein;

FIG. 9 is a chart of the self-assessed difference in the “lumpiness” of the cellulite of a group of subjects prior to and after completion of four weeks of use of an embodiment of a mechanical oscillation therapy device in accordance with the present invention;

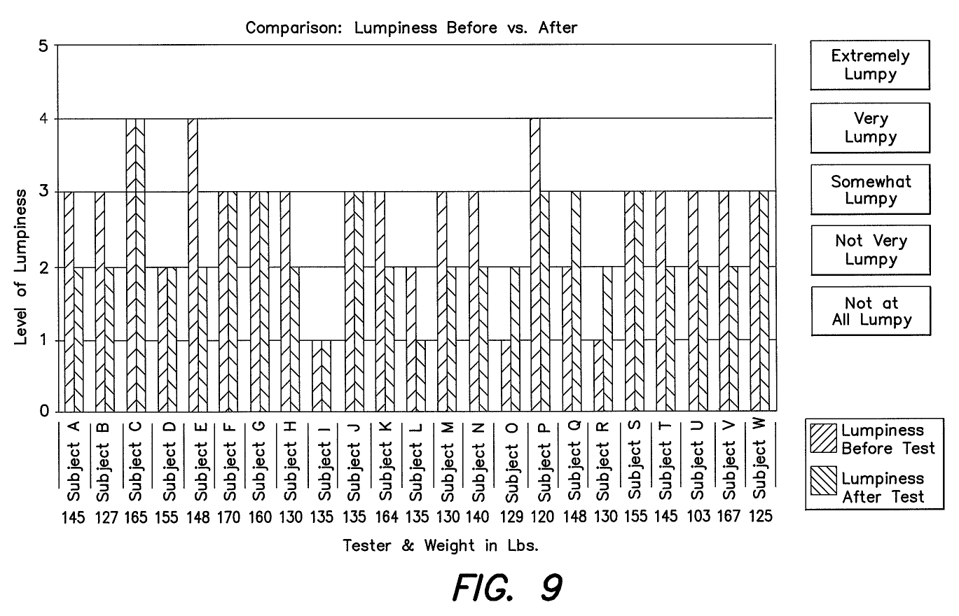

FIG. 10 is an illustration of photos of a woman viewed from the side before and after treatment with an example of a device according to the present invention; and

FIG. 11 is an illustration of photos of a woman viewed from the other side before and after treatment with an example of a device according to the present invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof, as well as additional items.

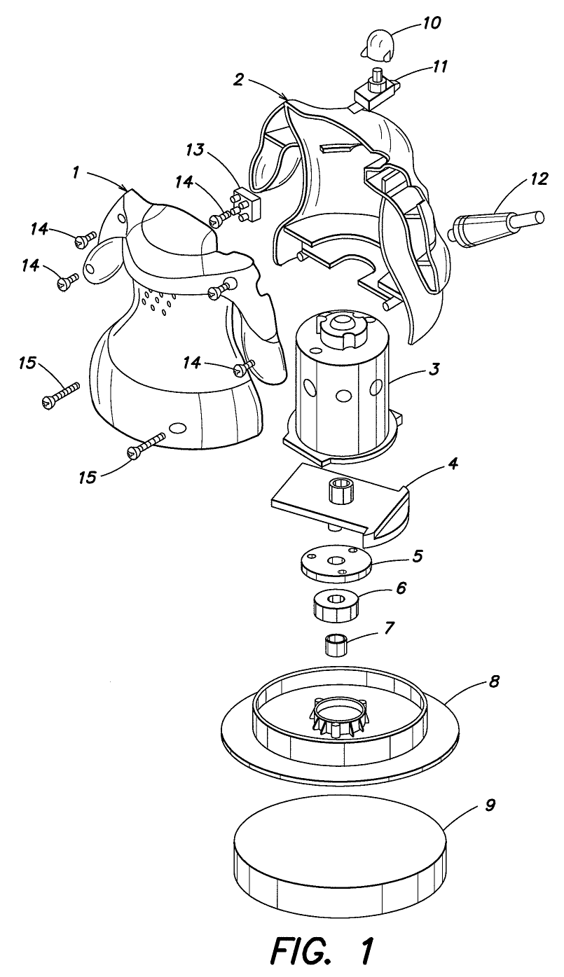

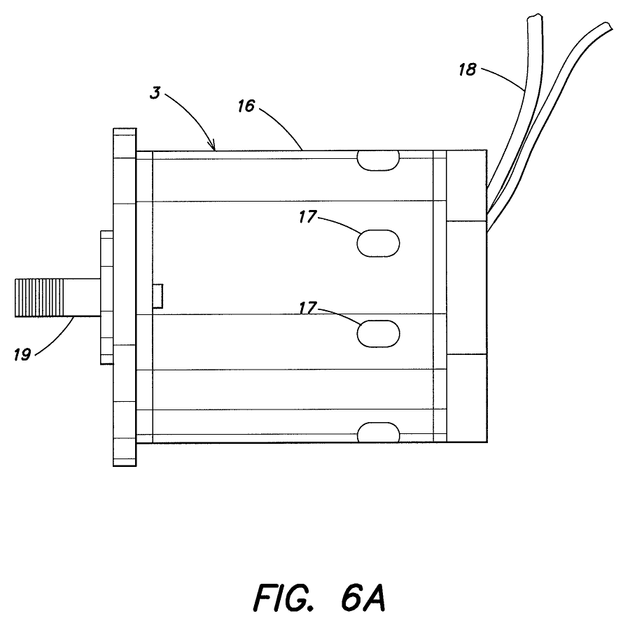

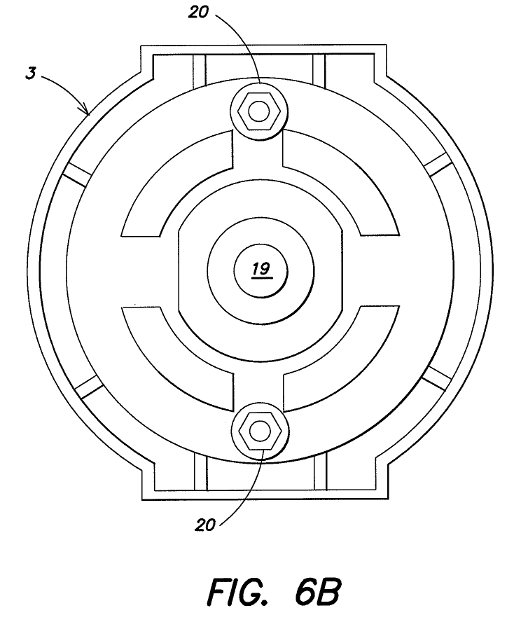

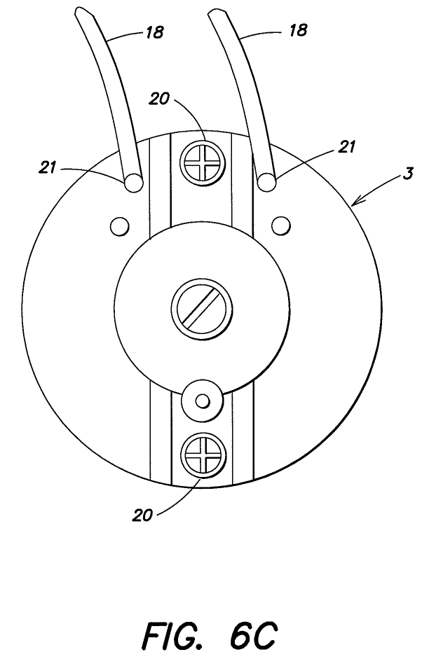

One embodiment of the mechanical oscillation treatment device is shown in FIG. 1. The device includes a housing (a body) with a front side 1 and a back side 2. Mounted within the housing is a motor 3. A counter weight 4 mechanically engages the shaft 19 of the motor (shaft 19 illustrated in FIGS. 6A and 6B). Mounted below the counter weight 4 are a cover bearing 5, an orbital bearing 6, and bearing 7. Bearings 6 and 7 are of a size and shape to fit into a circular receptacle in a pad frame 8. The pad frame 8 also includes mounting holes to which the cover bearing 5 may be attached. A buffer pad 9 is adapted to be mechanically mounted onto the pad frame 8. The buffer pad 9 is the contact surface of the device, and “buffer pad” and “contact surface” should be regarded as synonymous in this disclosure and in the claims. The term “contact surface” should also be understood to encompass any covering that may be present on the buffer pad 9. A switch cover 10 may be provided to cover an electrical switch 11 which is mounted on the device housing. Power is provided to the device through a power cable 12. A rectifier 13 may be included to convert AC power to DC power for driving the motor 3. Screws 14 and 15, or other fasteners, may be utilized to hold the housing front 1 and back 2 together and to secure the various components in the housing.

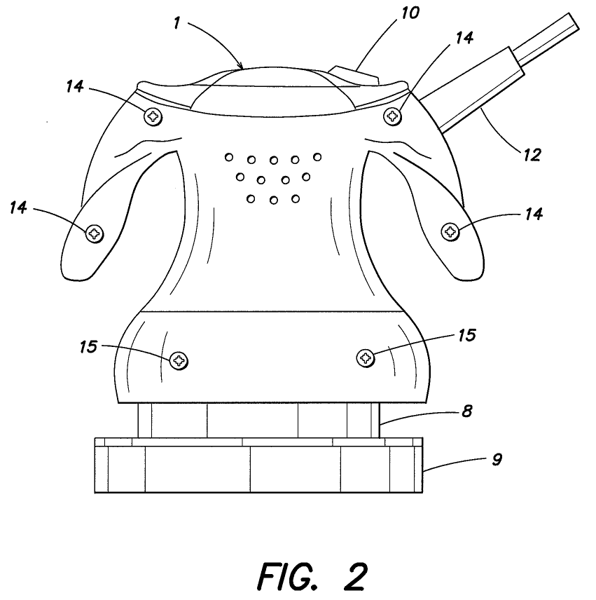

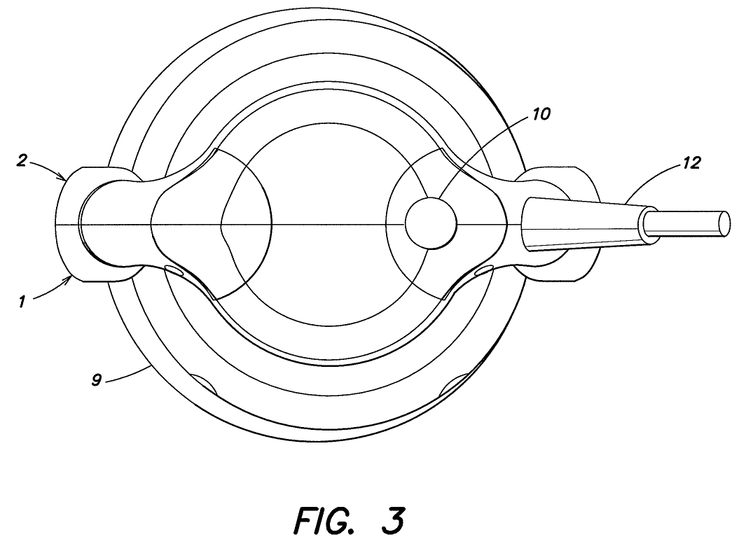

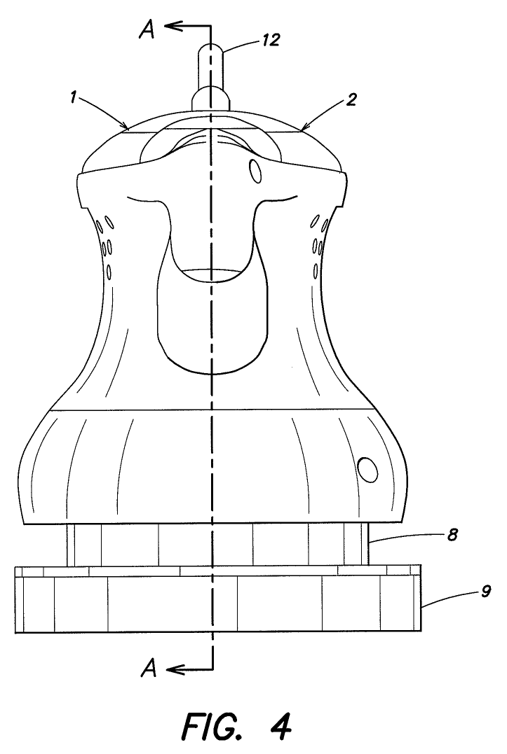

Side views of the embodiment of the oscillation therapy device of FIG. 1 are shown from two different sides in FIG. 2 and FIG. 4, while FIG. 3 illustrates a top view of the device of FIG. 1.

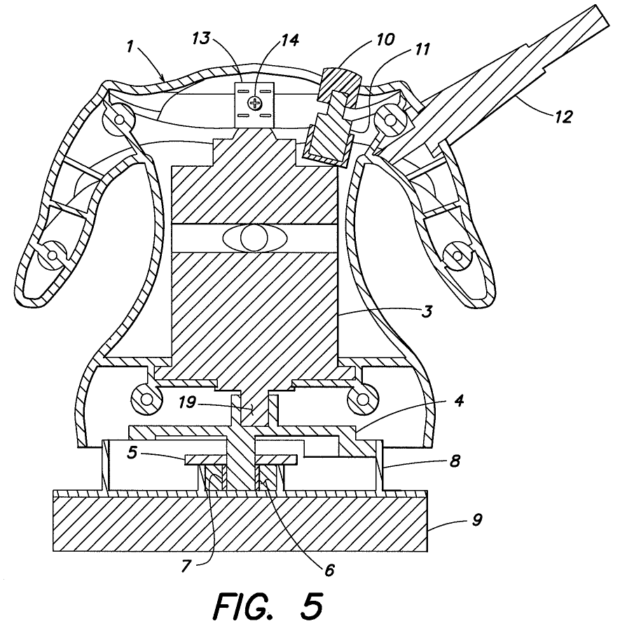

FIG. 5 illustrates a cross sectional view of the oscillation therapy device of FIG. 1 along the interface of the front and back sides of the housing along line A-A of FIG. 4. As can be seen in FIG. 5, the shaft of the motor 3 is coupled to the pad frame 8 through the counterweight 4. The shaft of the counterweight 4 is offset from the shaft 19 of the motor 3, and the pad frame 8 is free to rotate about the shaft of the counterweight 4 by means of the bearings 6 and 7. This allows the motor 3 to drive the pad frame 8 and the buffer pad 9 with a random orbital oscillating motion. The motor 3 may impart random orbital oscillating energy to tissue to which the buffer pad 9 is contacted by oscillating the pad frame 8 and the buffer pad 9 in a direction approximately parallel with the surface of the skin in an orbital motion.

The particular mechanical coupling between the motor 3 and the drive pad frame 8 illustrated in the embodiment according to FIG. 5 should not be considered limiting. Various methods of imparting random orbital oscillating mechanical energy from a motor to a drive pad are known in the art and should be understood to be encompassed by some embodiments of the present disclosure. (See, for example, U.S. Patent Publication 2008/0010759 to D. Scherl, entitled “RANDOM ORBITAL TOOTHBRUSH” and U.S. Pat. No. 7,318,768 to Uday Deshpande, et al., entitled “LOW PROFILE ELECTRIC SANDER” for descriptions of different random orbital oscillation drive mechanisms.)

The hand grips of the mechanical oscillation treatment device are not limited to the shape and size illustrated in the figures but may be of any number, shape, or size as might be appropriate for the various claimed embodiments or as may be selected by one skilled in the art.

One skilled in the art will also appreciate that the switch 11 and the switch cover 10 may be of various types and may be located on any of various alternate positions on the housing 1, 2, hand grips, or power cable 12 of the mechanical oscillation treatment device, or even separate from the device altogether.

The embodiment illustrated in FIG. 1 includes an electrical power cable 12 for providing power to the oscillation therapy device. Alternate embodiments of the device may forego such a cord and draw power from, for example, batteries internal to the device.

The buffer pad 9 may be constructed of or covered in a material that is adapted to frictionally engage human skin. The buffer pad may itself be constructed of or may include a covering (not shown)that may be constructed of any of a number of materials including such materials as polyurethane, cellular or foam rubber, cotton, lambswool, polyester, other plastics or fabrics, or any other material that would be recognized as appropriate to one skilled in the art. In some embodiments, the covering may be washable, in some embodiments sterile or sterilizable, and in some embodiments may be disposable. In some embodiments, the covering may be constructed of a soft material such as fluffed cotton, and in others may be a harder material, such as polyurethane.

The buffer pad 9 may be attached to the pad frame 8 by an adhesive or by a Velcro® hook and loop fastener or similar connection means. In some embodiments the buffer pad 9 may be removable from the pad frame 8. The buffer pad 9 may be retained on the orbital pad frame 8 by means of an elastic fitting. In some embodiments, the covering may be removable. The covering may be retained on the buffer pad 9 by means of an elastic fitting. In some embodiments, the motor 3 is adapted to impart random orbital oscillating motion to the buffer pad 9 and its covering.

When in operation, oscillating energy is applied to the buffer pad 9 of the mechanical oscillation treatment device. This energy may be of such a form as to drive the buffer pad 9 in random orbital oscillating motion. The imparted random orbital oscillating motion includes both rotation of the buffer pad about an axis and also rotation of the position of the axis of rotation of the buffer pad. This oscillating motion may occur at either a fixed or a variable frequency from about 2000 Hz to about 3000 Hz. In some applications, an oscillation frequency of about 2400 Hz may be desirable. The axis of rotation imparted to the buffer pad 9 may oscillate about a path having a diameter from about 8 millimeters to about 12 millimeters. In some applications, a diameter of about 10.2 millimeters may be desirable.

The buffer pad 9 or the pad frame 8 may include an integral pressure sensing transducer (not shown) or be coupled to a pressure sensor (not shown) that may measure pressure applied by the device. This pressure sensor may alternatively be located within the housing 1, 2 of the device. The pressure sensor may be coupled to a pressure indicator (not shown), so that the pressure applied to an area of tissue by the mechanical oscillation treatment device may be read by an operator, and may also be coupled to an automatic safety shutoff which may stop the motor of the device if the pressure applied is too great. This safety shutoff threshold may be adjustable or pre-set to a level of, for example, from about 10 pounds to about 15 pounds. In some applications, a contact pressure of from about 4 pounds to about 12 pounds may be desirable and the safety shutoff threshold would be set accordingly.

The buffer pad 9 or the pad frame 8 may also include a heating device (not shown). This heating device may be in the form of an electrical resistance heater coupled to the buffer pad 9 or the pad frame 8, or may be in the form of a hot air generator located in the housing 1, 2, or other heating device that may be known to one skilled in the art.

A suction generating device may also be included within or connected to the oscillation therapy device in order to apply suction through orifices that may be provided in the buffer pad 9 and/or the pad frame 8.

In some embodiments, the housing 1, 2 and hand grips may be integrally formed of a material such as plastic or metal. In other embodiments, the hand grips may be formed separately from the housing 1, 2 and attached by means of fasteners such as screws or other attachment means known to those skilled in the art.

In the embodiment illustrated in FIG. 5, a motor 3 may be positioned inside the housing 1, 2. The motor 3 may be fixed with the housing 1, 2 and connected to the counterweight 4 by means of a drive shaft 19. The motor 3 may be a universal motor operating on conventional 110V or 220V alternating electric current, or may be a direct current powered motor. The motor might also be adapted to run on battery power, or on any other power sources that might be appreciated by one skilled in the art.

The motor 3 is coupled to the counterweight 4, which in turn is coupled to the pad frame 8. Energy from the motor 3 may be transmitted through the motor drive shaft 19 and the counterweight 4 to drive the pad frame 8 and/or the buffer pad 9. The size and shape of the motor and drive shaft may be any size and shape that one skilled in the art might consider appropriate for the embodiments of the device claimed herein.

FIGS. 6A-6C illustrate an electric motor 3 that may be utilized in some embodiments of the mechanical oscillation treatment device described herein. The motor 3 includes a housing 16, which in some embodiments includes one or more vent holes 17. The motor 3 also includes a drive shaft 19. The drive shaft 19 may in some embodiments be threaded to facilitate attachment to a pad frame 8 as shown in FIG. 6A. As shown in FIG. 6B, the drive shaft 19 is located substantially at the center of the face of the motor 3. As shown in FIG. 6C, the motor 3 also includes one or more electrical wires 18 connected to the motor at electrical connections 21. These wires may exit the body of the mechanical oscillation treatment device through power cord 12. In some embodiments, one or more capacitors and/or inductors may be present in the electrical circuit including electrical wires 18. The motor is held together in part by fasteners 20, which in some embodiments may be bolts that pass through the body of the motor. The motor is a DC motor which operates at 115 volts. The motor has a no load speed of 3304 rpm, at which it draws 0.149 A of current. The motor is rated to supply 1,000 gm-cm (13.89 oz-inch) of torque at 3,162 rpm, drawing a current of 0.439 A. The motor has a stall point torque of 23,239.6 gm-cm (322.74 oz-inch) at which it draws 6.9 A of current.

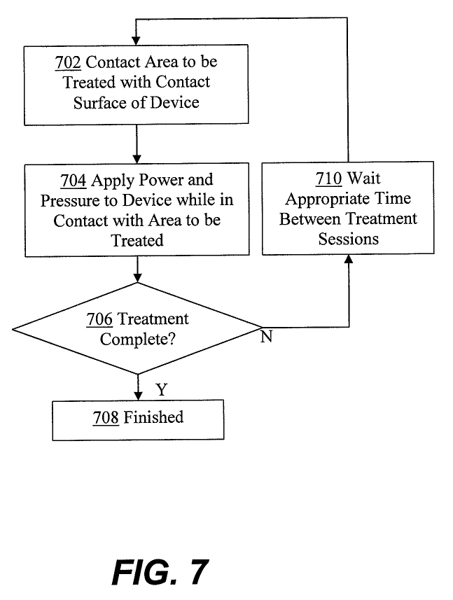

Embodiments of the mechanical oscillation treatment device described herein may be used to apply mechanical oscillating energy treatment to a person and may be used in the treatment of cellulite. Treatment with the mechanical oscillation treatment device described herein may include individual treatment sessions performed on, for example, a daily, or a twice daily basis. Treatment sessions may be repeatedly performed over a sufficient number of days to provide a desired reduction in the appearance of cellulite. FIG. 7 is a flowchart of one such treatment method. The method may include a step 702 of contacting the surface of an area of tissue to be treated with the contact surface of the treatment device, and a step 704 of applying power to the device to drive the motor 3, thus imparting oscillating mechanical energy to the area of tissue to be treated. The oscillating mechanical energy may in some applications be random orbital oscillating mechanical energy.

In some methods of treating cellulite with an embodiment of a mechanical oscillation treatment device as described herein, energy may be applied to each of one or more areas of tissue to be treated for a period of between 1 and 15 minutes per treatment session. In some applications, a treatment time of between 5 and 10 minutes per treatment area may be desirable in each of one or more treatment sessions. It may also be desirable in some applications to have multiple treatment sessions per day. In the method illustrated in FIG. 7, the decision as to whether additional treatment sessions would be required would be made at step 706. In step 706, a decision may be made as to whether additional treatment sessions would be desired in the same day, and/or whether treatment should continue for additional days in order to achieve desired effects and/or to maintain a desired appearance. The nature and amount of cellulite in the tissue to be treated, as well as the tolerance to the treatment of the person treated, and the stature of the person to be treated may be factors in determining the aggressiveness of the treatment and the duration of the treatment in step 704, or the time between treatment sessions in step 710, the total number of treatment sessions required, or the number of days over which treatment should take place. If no further treatment sessions are deemed required, then in step 706 the treatment may be considered complete and the method according to FIG. 7 may be considered finished (step 708.)

While applying oscillating energy with an embodiment of a mechanical oscillation treatment device as described herein, some treatments may be performed while applying pressure with the device to the area of tissue to be treated in step 704. Application of a pressure in the range of from about 4 pounds to about 12 pounds between the device and the area of tissue to be treated may be desired in some methods.

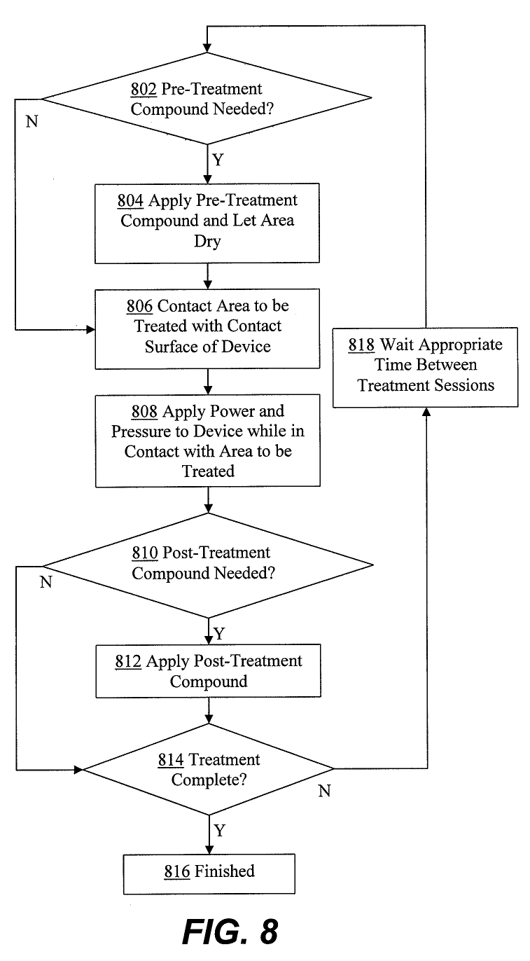

In some methods according to the present invention, a moisturizer or cellulite treatment enhancement compound may be applied prior to, or after, treatment with the mechanical oscillation treatment device, as is illustrated in FIG. 8. In step 802 of the method illustrated in FIG. 8 it is determined whether some form of pre-treatment compound, such as a moisturizer or similar compound is desired. If such a compound is desired, it may be applied in step 804 and allowed to absorb into the area or areas of tissue to be treated until the treatment area or areas are dry. In some methods, about 30 minutes or more may be allowed after application of the compound for the compound to absorb into the tissue to be treated until the treatment area is dry. In steps 806 and 808, an area or areas of tissue may be treated with the mechanical oscillation treatment device in a similar manner as in steps 702 and 704 of the method illustrated in FIG. 7.

In some methods a treatment compound may be applied to the tissue treated after treatment with oscillating mechanical energy is completed. The decision as to whether such a compound is to be applied may be made in step 810 of the method illustrated in FIG. 8. The compound may be applied in step 812. This compound may be, for example, a moisturizer. In some methods, the individual undergoing treatment may bathe prior to the application of this compound.

In step 814, it may be determined whether or not treatment is complete, such as may be done in step 706 of FIG. 7. In step 814 of FIG. 8, like in step 706 of FIG. 7, the decision may be made as to both whether further treatment should be performed on the same day or on subsequent days as part of a longer term treatment process, whether the treatment process as a whole has been completed, or whether maintenance treatments should be performed at a later date. Steps 816 and 818 of FIG. 8 are analogous to steps 708 and 710 of FIG. 7, respectively.

In some methods, treatment with a mechanical oscillation treatment device according to one or more embodiments disclosed herein is performed in the morning. This may be desirable because in some methods utilizing some embodiments of the mechanical oscillation treatment device, treatment results in exfoliation and/or includes the application of compounds to areas of tissue that a treated individual may wish to wash off by bathing or showering after the treatment session is concluded.

In some methods, after a treatment or series of treatment sessions has been performed, maintenance treatments may be performed in order to maintain the reduction in cellulite and desired appearance. The time between maintenance treatments and the aggressiveness of such treatments may be determined by factors such as the amount of time it takes for the appearance of cellulite to return, as well as the tolerance to the treatment of the person treated. Maintenance treatments may follow the methods illustrated in FIG. 7 and/or FIG. 8, or may follow other, similar methods.

### EXAMPLE I

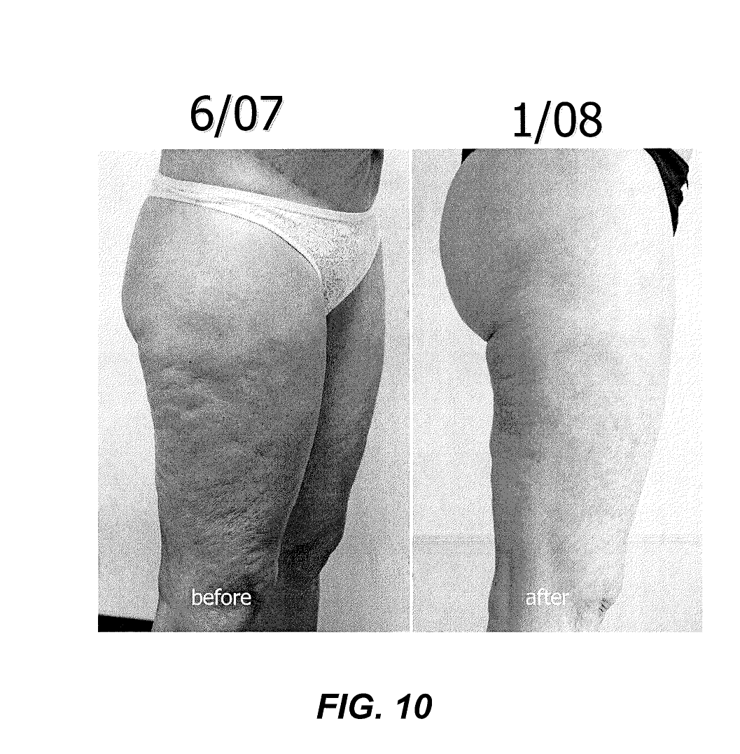

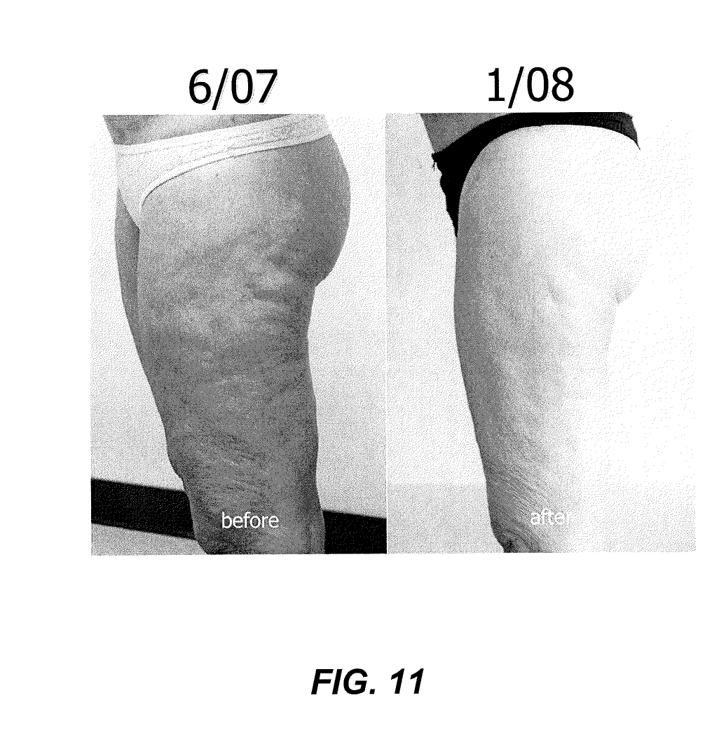

The photos illustrated in FIGS. 10 and 11 are of a woman before and after 6 months of treatment with the device of FIG. 1 for 10 minutes twice a day, according to the protocol described in Example II.

### EXAMPLE II

After plugging the power cord into the outlet and holding the device securely with both hands at the handles, the contact surface is placed directly onto dry skin in the area of the body to be treated, such as the thigh. Press the button on the handle to activate the device. While applying pressure the device is moved in a circular and side to side motion, up and down and all around the affected area, maintaining constant motion while firmly massaging the tissue.

Treat each desired area for preferably about 3-5 minutes, and up to about 10 minutes at a time, twice per day. If desired, a moisturizing cream or lotion can be applied. Uptake of the compound will be enhanced due to the increased tissue perfusion after treatment.

### EXAMPLE III

In 2008, twenty three subjects employed the device of FIG. 1 for four weeks, using it twice a day on their areas of cellulite. The graph shown in FIG. 8 illustrates the self-assessed difference in the “lumpiness” of the individuals' cellulite before and after completion of the testing. Lumpiness of the individuals' cellulite was determined by the individuals running their hands over their areas of cellulite and assessing how lumpy the areas of cellulite felt on a scale of one to five, with five being “Extremely Lumpy” and one being “Not at all Lumpy.” As can be seen in the chart of FIG. 8, twelve (52%) of the individuals noticed an improvement, defined as a decrease, in the lumpiness of their cellulite after using the device. Eight individuals (35%) did not notice a difference in the lumpiness of their cellulite. Three individuals (13%) noticed an increase in the lumpiness of their cellulite. The reduction in the level of lumpiness of the individuals' cellulite was statistically significant. The average lumpiness value pre-treatment was 2.74. The average lumpiness value post-treatment was 2.30. This difference had a statistical p value of 0.013. Further, sixteen (70%) of the individuals reported a difference in the appearance of their cellulite. This data shows that the level of lumpiness of cellulite can be improved in about 50% of individuals and the appearance of cellulite can be improved in the majority of individuals after four weeks of treatment with an embodiment of the device disclosed herein.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1) A mechanical oscillation treatment device comprising:
a body;
a motor positioned within the body;
a drive shaft coupled to the motor, wherein the motor is adapted to power the drive shaft; and
a contact surface coupled to said drive shaft, the contact surface adapted to frictionally engage human skin,
and wherein the motor and drive shaft are adapted to drive the contact surface to impart an orbital oscillating motion to the contact surface.

2) The mechanical oscillation treatment device of claim 1, wherein the motor and drive shaft are adapted to impart a random orbital oscillating motion to the contact surface.

3) The mechanical oscillation treatment device of claim 2, wherein the motor and drive shaft are adapted to impart random orbital oscillating mechanical energy to the contact surface at a frequency in the range of from about 2000 Hz to about 3000 Hz.

4) The mechanical oscillation treatment device of claim 3, wherein the motor and drive shaft are adapted to impart random orbital oscillating mechanical energy to the contact surface at a frequency of about 2400 Hz.

5) The mechanical oscillation treatment device of claim 2, wherein said random orbital oscillating motion has an orbit diameter of from about 8 mm to about 12 mm.

6) The mechanical oscillation treatment device of claim 5, wherein said random orbital oscillating motion has an orbit diameter of about 10.2 mm.

7) The mechanical oscillation treatment device of claim 1, further comprising a pressure sensor coupled to the contact surface.

8) The mechanical oscillation treatment device of claim 7, wherein the pressure sensor is coupled to a pressure indicator.

9) The mechanical oscillation treatment device of claim 7, further comprising a shutoff configured to be activated when the pressure sensor reads a pressure exceeding a pre-set pressure value.

10) The mechanical oscillation treatment device of claim 9, wherein the pre-set pressure value is in a range from about 10 to about 15 pounds.

11) The mechanical oscillation treatment device of claim 1, wherein the contact surface comprises a multilayered component including a rigid disk, a layer of cellular rubber, and a surface cover substantially composed of at least one of polyurethane, cellular rubber, cotton, lambswool, and polyester.

12) The mechanical oscillation treatment device of claim 1, wherein the contact surface comprises a polymeric material with at least one of massaging protrusions and bristles.

13) The oscillation therapy device of claim 11, wherein the surface cover is washable.

14) The mechanical oscillation treatment device of claim 11, wherein the surface cover comprises an elastic band adapted to facilitate securing the cover to the device.

15) The mechanical oscillation treatment device of claim 1, further comprising a heating means in thermal contact with the contact surface.

16) A method of applying mechanical oscillating energy to human tissue, said method comprising:
contacting an area of tissue characterized by cellulite with the contact surface of an mechanical oscillation treatment device; and
applying power to the motor of said device while said contact surface is in contact with an area of human tissue to apply orbital oscillating energy to the tissue by oscillating the contact surface in a direction approximately parallel to a surface of the tissue in an orbital motion to induce shearing and stretching forces in the tissue.

17) The method of claim 16, wherein the orbital oscillating energy applied to said tissue is random orbital oscillating mechanical energy.

18) The method of claim 16, performed for about 5 to about 10 minutes per day on each of one or more areas of tissue.

19) The method of claim 18, performed twice per day.

20) The method of claim 19, performed for a time period in the range of from about 3 weeks to about 6 months.

21) The method of claim 16 further comprising applying a contact pressure in a range of from about 4 pounds to about 12 pounds between the device and the area of tissue.

22) The method of claim 16, further comprising applying a treatment enhancement compound to the area of tissue after contacting the area of tissue with the contact surface of the device for treating cellulite.

23) The method of claim 22 wherein the treatment enhancement compound comprises at least one of an analgesic, a moisturizer, and a warming solution.

24) The method of claim 16, further comprising applying a treatment enhancement compound to the area of tissue prior to contacting the area of tissue with the contact surface of the device for treating cellulite.

25) The method of claim 24, wherein the treatment enhancement compound is applied to the area of tissue no less than about 30 minutes prior to contacting the area of tissue with the contact surface of the device for treating cellulite.

26) The method of claim 16, further comprising one or more maintenance treatments.
